(19) United States
(12) Patent Application Publication
(10) Pub. No.: US 2014/0276088 A1
(43) Pub. Date: Sep. 18, 2014

(54) Illumination Optics for a Visible or Infrared Based Apparatus and Methods for Viewing or Imaging Blood Vessels

(71) Applicant: Steven H. Drucker, Oakland, CA (US)

(72) Inventor: Steven H. Drucker, Oakland, CA (US)

(21) Appl. No.: 13/843,958

(22) Filed: Mar. 15, 2013

(51) Int. Cl. A61B 5/00

(52) U.S. Cl. CPC A61B 5/489; A61B 5/0082; A61B 5/0077; A61B 5/0075
USPC 600/473; 600/479; 600/476

(57) ABSTRACT

The illumination apparatus and methods described herein increase the depth of the illumination's tissue penetration, help minimize surface reflections and back-scatter for a non-contact camera based imaging system thus providing increased tissue-structure contrast and more information about the structures beneath the surface. It does this by using one or more of the following techniques:
using optics to provide radiation which hits the surface at or near 90 degrees for better tissue penetration;
using optics and radiation source placement to control the angular distribution of light from surface vertical to minimize surface specular reflection and subsurface reflection;
removing some surface light reflection through patterning the intensity of the light source thus increasing contrast in areas of no or low direct irradiation;
synchronously with respect to camera frames or through user selection, switching on and off light sources which has the effect of 1) dynamically changing the overall angular distribution of light thus changing surface level reflectance; 2) revealing and through processing removing unwanted patterning caused by optical defects or contaminants on optical surfaces or surface hair; 3) moving illumination patterns to permit contrast enhancement in all areas of the surface.

### CROSS-REFERENCE TO RELATED APPLICATIONS

This application can be used to improve the device in application B622918 of whom I am the named inventor and which is incorporated by reference.

### FIELD OF THE INVENTION

The present apparatus and methods relate generally to an improved system or device and method for imaging or visualizing blood vessels or other body tissue to facilitate accurate placement of a needle or other elongate instrument in blood vessels or other body tissue.

### BACKGROUND

Inserting an intravenous line (IV) requires knowing where a suitable vein or other blood vessel is located and how large a needle the vein will support. For non-Caucasian individuals, females, small children and neonates, the elderly, obese individuals, those who have acute medical problems, and others, veins may not be visible. Individuals who exhibit more than one of the above traits often have veins that can be very difficult to find and may require multiple attempts to insert an IV.

In these difficult cases, caregivers have traditionally resulted to palpating the area around a potential vein site rather than locating a vein visually. When dealing with sick individuals, or when working in an area where spread of contagious diseases is likely, such as a hospital, this may not be possible. Blood pressure may be too low, or a vein may be buried too deeply to find by touch. Regulations designed to halt the spread of MRZA or other contagion may require the caregiver to wear gloves, severely diminishing touch sensitivity and the chances of finding a suitable vein. Problems with inserting a needle into a vein can result in escalation procedures which require additional personnel to become involved or a central line to be inserted by surgery adding to infection risk and compromising patient safety. In all cases, critical time and resources are wasted, patient discomfort is increased, and patient care is compromised.

Any device on the market which seeks to use visible or infrared light to image structures beneath the surface of the skin suffers from the diffusing properties of skin and tissue, which limits depth of penetration. This can readily be seen by shining a laser pointer on the web of tissue between the thumb and forefinger. At the entry site, there is a round dot composed of the reflection of the laser directly from the surface of the skin. On both sides of the hand, there is a diffuse glow where the light from laser beam exits after being scattered within the skin and body tissue. Both the initial skin reflection and the internal scattering of light obscure structures beneath the skin. All of the devices on the market suffer to a greater or lesser degree with this problem of skin penetration.

In individuals, veins are located at different locations and depths and individuals have different thicknesses of skin which incident radiation needs to penetrate in order to illuminate the vein. When this inventor first started designing a portable vein viewer, a very simple device was built: just a single infrared LED, a camera, and a display. The picture showed a bright spot where the LED was focused, and a nimbus of radiation around that spot. If a vein were present within, it appeared as a darker line within LED lighted area on the display and was simpler to discern outside the central spot. The optics quickly evolved to a device with four larger angle emission LEDs at the corners of a square to produce more even illumination, with the video camera in the square's center. One problem with this approach was specular reflection, and one remedy was to move the illumination off axis. This approach was described in the patent application referenced above and can also be seen in Figures in other patents awarded: 4817622, 5519208, 6032070. However, neither the approach of LEDs at the corners of a square nor the approach of an angled light beam revealed deeper veins. After experimentation, it became apparent that the angle of the incident radiation partially determined the depth at which veins could be seen. There is a correlation between the radiation angle of incidence and the amount of light scattered at a given depth. The more normal to the surface, the lower the scattering near the surface. Once the angle is less than 5 to 10 degrees off normal, no further improvement is found. This makes sense: each layer of skin and each cell membrane and internal structure is a potential scattering sight, at which Rayleigh scattering can take place. Rayleigh scattering occurs when the wavelength of radiation is about the same as the particle size that the radiation passes through. As the angle of incidence decreases, the number of scattering sites per unit of depth increases, decreasing contrast both due to the scattering above the vein, and less light reaching the vein. The AccuVein AV300, a device for projecting vein position on the skin suffered from this problem as can be seen looking at the vein changes between the center and edges on pictures in AccuVein's sales literature.

The optics and methods described herein help improve the contrast between tissue and blood vessels and increase the depth at which a vein can be recognized. The basic principle behind blood vessel detection using selected wavelengths of light is that hemoglobin within a blood cell selectively absorbs light radiation in certain spectral bands whereas normal tissue does not. Therefore, a vein filled with blood, which contains hemoglobin, will appear darker than the surrounding area. However, as mentioned in [0006] above, Rayleigh scattering and direct reflection of light from the skin surface significantly reduce the contrast making deeper veins a significant challenge. Also, light penetration varies with epidermal thickness, adding yet another variable to be contended with.

The optics described herein solve this problem by providing incident radiation near normal to the skin surface and by using other methods to increase contrast detailed in the sections below. Luminetx, now Christie Medical, had the first commercially successful vein viewer on the market. This vein viewer had excellent skin penetration and achieved that result by having a patented uniform illumination source that was about 30 inches from the skin surface. AccuVein, which entered the market latter with a hand-held device projected a laser beam whose angle increased as it moved from the center of the picture to edge, resulting in a poorer quality vein contrast and vein depth as one moved from the center of the vein projection to the edge of the vein project. This problem was definitely not foreseen by the original engineers. Hence, deliberately including optics that provides incident illumination at 90 degrees to the surface at short distances is a major non-obvious state-of-the-art improvement. Contrast can also be increased through other non-obvious mechanisms detailed below.

When this inventor reviewed prior work after completing the design, the only patent that that focused specifically on illumination for improving vein contrast in a non-contact system was Zeman's patent 6556858, Diffuse infrared light imaging system. Zeman, a founder of Luminetx, was particularly concerned about revealing blood vessels underneath subcutaneous fat. In his patent, he states, “However, due to the reflective nature of subcutaneous fat, blood vessels that are disposed below significant deposits of such fat can be difficult or impossible to see when illuminated by direct light, that is light that arrives generally from a single direction. The inventor has determined that when an area of body tissue having a significant deposit of subcutaneous fat is imaged in near-infrared range under illumination of highly diffuse infrared light, there is a significantly higher contrast between the blood vessels and surrounding flesh than when the tissue is viewed under direct infrared illumination.” Zeman's solution of diffuse radiation to achieve fat penetration and this inventors solution of near normal radiation to achieve greater depth of penetration appear to be at odds. And, unlike many patents, Zeman's patented approach to a diffuser works in a successful product so it needs to be discussed seriously in this patent and also serves to further illuminate why the apparatus and methods claimed in this patent are unique.

First, assume that Zeman's diffuser produces a light output that radiates evenly into a hemisphere as claimed. Further, from their current promotional video, light exits from a square roughly an inch on side (or less) and illuminates an area approximately 1.25″×2.5″ at a controlled distance of 9″ to 10″ (when the device is at the correct height, projected characters are in focus.) Luminetx original device had a source even further away from the patient. The maximum angle of the “diffused” light hitting the skin's surface can be calculated as roughly 11 degrees with typical radiation on the order of four to eight degrees. This qualifies as being “near normal” for which the apparatus described herein seeks to achieve. Providing “near normal” irradiation is not discussed in the Zeman patent and was not obvious until the actual device was examined. Furthermore, this inventor needed a new approach since it is being applied to a device that is almost two orders a magnitude smaller than the original VeinViewer. Furthermore, this inventors apparatus and methods include off axis source(s) which increase the angular dispersion of the beam, achieving the same effect as Zeman's device without the diffuser. Deliberately using off axis elements is not obvious.

As can be seen from looking at patent application B622918, this inventor is concentrating on a portable device that is much closer to the skin of the patient than any current device on the market. This makes the demands for the optics and optical path much more demanding than in other devices. Yet, the invention described in this application would also improve contrast and depth of vein detection on devices with longer optical paths or allow them to be miniaturized further.

### BRIEF SUMMARY

This patent details various optical systems, devices and methods for illuminating blood vessels or other body tissue to increase depth of visible and/or infrared radiation penetration and improving the contrast between vein and non-vein areas.

It describes an illumination system that is either reflective or transmissive or a combination which provides incident illumination radiation that is near normal to the surface even if the imaging system is close to the surface. In one embodiment it allows the operator to view the insertion site even when the imaging system is close to the surface as in a hand-held device.

It describes an illumination system whose radiation may be patterned into lines or distributed small areas, so that direct reflection of incident radiation from the skin is limited to specific areas allowing contrast improvement in areas that are illuminated mainly by light scattered in the tissue.

It describes an imaging system whose source radiation location may vary in order to move an illumination pattern in a predefined way across the skin thereby increasing resolution and contrast and/or to help remove artifacts caused by the illumination, illumination pattern, and/or the imaging system itself.

It describes an imaging system whose source radiation angle of incidence may dynamically vary in order to remove of improved specular reflection and improve depth of penetration.

### BRIEF DESCRIPTION OF THE SEVERAL VIEWS OF THE DRAWINGS

FIGS. 1A & 1B illustrate top and side views of typical apparatus using a reflective collimation technique, along with a reference illumination path

FIGS. 2A, 2B, & 2C illustrate an extended source with alternative collimation optics

FIGS. 3A & 3B illustrate using a collimator plate

FIGS. 4A & 4B illustrate patterning the incident illumination on the skin

FIG. 5 illustrates a contrast enhancement for a deeper vein using a patterning method

FIG. 6 illustrates one way of moving the incident illumination patterning across the skin by using more than one illumination source.

### DETAILED DESCRIPTION

The optical systems and methods described here-in increase contrast and depth of skin penetration to reveal veins that can not be found by manual methods. Three main approaches are taken that can be used independently or together to achieve this result:
A. Providing incident radiation in one or more wavelengths of hemoglobin absorption that is normal or near normal to the surface. Using a reflector based system, preferred embodiment, or a lens based system, single or multiple radiation sources can be used, each illuminating the whole scene through the optical systems. The further the amount of deviation of the radiating source from the optical system's point of focus, the greater the greater the angular spread of the near normal radiation on the surface being illuminated. This reduces both specular from the surface and direct reflection from more reflective objects between the surface and the vein such as adipose tissue. Furthermore, the angular dispersion can be controlled by design or manipulated on a frame by frame basis through turning off and on different sources. Turning off one or more sources of the extended source also changes the radiation pattern, allowing a radiation angle that has a large specular reflection component to be removed. A method is provided to achieve this goal.
B. Providing a static pattern of incident (normal) radiation which includes areas of full illumination and areas with little or no direct illumination. Areas with full illumination and no illumination would be sampled with different criteria and by sampling for contrast changes within each area that would possibly indicate a vein in that area Note that areas with no incident radiation would be more sensitive to deeper veins since no skin reflection would obscure fainter veins, and deeper veins would have less scattering above them. This is in complete contrast to the Luminetx patents for producing a uniform surface illumination and hence non-obvious. In the no surface illumination case, the light scattered within the tissue serves as the illumination source. Such a pattern could be composed of spaced lines of light and dark areas, or a two dimensional pattern of light and dark spaces. The major criteria is that there be only minor aliasing effects between the veins and the sampling illumination pattern. Any such effects can be minimized using the technique in C) below.
C. Deliberately changing the source location of the incident light. In combination with A) above, this can be used to remove shadows caused by irregularities in the light source or other optical issues and possibly certain surface blemishes such as wrinkles or hair, which are orientation dependent. In combination with B) above, moving the pattern allows for additional vein resolution and detection by sampling at different low surface illumination points. In both of these cases, processing to determine the scene differences would be employed. In the case of A) above, optical imperfections can be readily removed since they always will exist in a known location within the illuminated field and could be removed by changing the chosen source. For instance, should the sources be located to provide a +−1.5 degree shift in incident angle, small enough so that the internal scattering pattern would be essentially unchanged, and, as an example, if the offending defect were 2″ away from the surface, the defect would be moved 0.1″, sufficient for easy removal through software. Likewise, any patterns would be shifted by a similar amount, and through proper design, a surface area that was illuminated using one source could be shifted into coincide with a previous dark area from another source.

FIG. 1A, Top View Reference System, shows a typical apparatus as a reference device in which the illumination subsystem exists. It consists of the following major subsystems:
(1) An electronics subsystem which holds the processing elements, power, and other such components.
(2) A display subsystem which can be a separate LCD, or a direct skin projection subsystem.
(3) A source of optical radiation, as represented by the dashed lines, used to illuminate the reflector (4) and after reflection illuminates the skin area being imaged.
(4) A reflector shaped to take the incident radiation from (3) and project it on to the skin such that the radiation hits the skin near a 90 degree angle. The reflector can be a spherical subsection or optics specifically designed to optimize light angle.
(5) The skin surface being scanned for veins, which is underneath the reflector in the area between the dashed lines
(6) A camera with appropriate wavelength filters used to pick up the reflected light from the skin.

FIG. 1B, Side View Reference System, shows a side view of the illumination subsystem of 1A with two of the light rays (7) traced. A secondary mirror, (8) may be used to make the optics more compact. If a spherical subsection is used for the reflector, its radius can be calculated by a competent optical engineer based upon its size and distance from the light source. The reflector can be manufacture from polished metal, metalized plastic, or be made from transparent plastic such as acrylic with an optically reflective coating (9). A dichroic reflective coating which reflects near infrared light allows visible light to pass through so an operator an unrestricted view of the skin surface below. Note that the reflector is tilted at an angle (A) with respect to the surface. This angle is derived from the apparent angle, (B) of the source (3) with respect to the center point of the reflector (4). The source needs to be outside of the scanned area (5) or it will show up as a very bright, undesirable artifact in the vein image. Angle A (10) is angle B/2 (9). In place of a dichroic filter, a coating (9) that reflects only polarized light can be used with a source (3) that is similarly polarized. This has an advantage if visible rather than infrared light is used. Candidates for the extended source (3) are LEDs or OLEDs with the desired wavelength and with an emission angle such that the reflector (4) is fully illuminated, or a laser with the desired wavelength whose beam has been expanded to fully illuminate the reflector (4).

FIG. 2A, Extended LED Source with Beam Dispersion shows a cross section of an alternative scheme with multiple individual light sources possibly with a lens (11) or internal reflector which controls the beam dispersion.

FIG. 2B, Top View Extended LED Source Example shows an array of LEDs (3) whose spacing and number can be designed to provide the desired angular dispersion of the combined beam.

FIG. 3B, Side View of Extended Source with Collimation Lens shows an alternative to the reflector approach shown in FIGS. 1A and 1B. As in the case of the reflector (9), each source fully illuminates the optical collimation element, in this case (33), whose exit radiation is nearly perpendicular to the lens element (33). However, each off center source provides parallel but not perpendicular rays (34). This added dispersion diminishes specular reflections.

FIG. 3A, Top View Collimation Plate, and FIG. 3B, Side View Collimation Plate, shows yet another method of achieving the same objective of producing light normal to the skin surface. FIG. 3A shows a light source or light source(s) which produces a wide, narrow beam (7) which interacts with a plate (14) with multiple prisms (13) along its top.

FIG. 3B shows the prisms in more detail, each with a reflective coating (15). These prisms are angled to reflect lines of light normal to the surface. Depending on the dispersion of the light source, the light could project narrow lines or varying intensity lines on the surface as shown in FIG. 4A. Note that other variations of this same theme can be conceived.

FIG. 4A, Spaced Line Illumination, shows a simple example of a pattern of incident radiation on the surface of the skin composed of illuminated lines (16) with areas of little or no direct illumination (17) and are illuminated mainly by internally scattered light. All of the tissue gets some illumination from nearby lighted areas since directly illuminated tissue scatters light into adjacent tissue without direct light.

FIG. 4B, Patterned Illumination, shows a simple example of patterned illumination where the dark areas (17) of the skin (5) are not directly illuminated and the light area (16) is. Note that the indirect illumination comes from not just the local light area, but also from extended light areas around other dark areas which could be a half an inch or more away.

FIG. 5, Interaction Between Illumination and Two Veins shows an example of the contrast enhancement using this technique. A vein that is close to the surface (18) or has a large cross section absorption area appears in both the area directly and indirectly illuminated whereas a deeper vein (19) is not visible under direct illumination but is revealed under indirect illumination of the incident radiation scattered by the tissue.

The two criteria for either of these examples to work is that the projected patterns on the camera sensor be imaged by the sensor, at least 3 times the size of camera pixel, and that they be sufficiently smaller than the minimum vein thickness or larger than the maximum vein thickness such that major aliasing effects to not occur. In addition the camera's sensor must have good dynamic range and a high signal-to-noise ratio.

Irradiation patterning can be achieved in three basic ways:
Absorbing the light in the areas where it is unwanted. In general this is not the preferred embodiment since optical systems generally suffer from a shortage of radiation. Absorbing the light either adds to the power requirement by requiring the source to be brighter or adds to the sensor requirements by requiring a higher signal-to-noise ratio.
Passing the light through an additional optical system such as a micro lens array or a cylindrical lens array or doing the equivalent using a laser and hologram. Again this is not the preferred embodiment unless an array of sources is used as detailed in FIGS. 2A & B in which case only the specifications of the lens array change. Otherwise, this approach, due to the additional component and system complexity is not preferred.
When using the spherical reflector approach, add to the design of the mirror used to fold the optical path to include either a stamped pattern to shape the light output or Fresnel optical elements to meet the desired patterned radiation specification.

FIG. 6, External Object Shift with Two Sources, shows a simple optical system consisting of two illumination sources (22) (24) a focusing reflector (4) and various ray traces. The sources have a smooth radiation angular distribution pattern which serves to illuminate the reflector (4). The reflector is designed in such a way that with a source centered at its midpoint (23), the reflected radiation is perpendicular to the surface (5) it strikes. Such a reflector can be constructed from a reflective spherical surface canted, if necessary (shown in FIG. 1B), to compensate for a source off center with respect to the reflector. Other reflectors are possible. A spherical surface can be further optimized to remove spherical aberration if desired. With two sources equidistant from the center point, the radiation strikes the surface slightly off perpendicular as shown in the two rays (26) (27) corresponding to light sources (24) and (26) respectively. If there is a blemish, defect, or pattern on the reflector (20), its position will be shifted on the surface due to the shift in source (24) from optical center as shown by rays (29) to a new apparent surface position (28). Likewise, using the other source (22), there is a shift in apparent position of the object in the other direction. However, none of the structures under the surface will appear to have moved. Using a computing element, a comparison can be made of the two pictures and only stationary objects need be displayed. Note that in the case where the optical system is moving with respect to the surface, that translation in position can also be separately compensated for in the computing element, and only the objects under the surface displayed. Should a pattern or object be located other than on the reflector such as on surface (21) the displacement still occurs as shown in ray traces (33) with the resulting (in this case magnified) object positions 31 and 32. Since the position of the radiation source is different any fixed patterns in the optics will move with a change in source, while the radiation hitting the surface will still be essentially normal to the surface. By design, the degree to which the radiation varies from perpendicular can be held to less than +−5 degrees and with an average of less than +−2.5 degrees, so as not to significantly change the depth of penetration or radiation scattering characteristics. By turning on the one light source at a time synchronously with the start of a new camera frame, the radiation pattern is changed as desired in different frames. As previously stated, this allows further processing to remove undesirable artifacts. It also allows the illumination pattern on the skin to be shifted, so that areas in a previous frame that had direct illumination now receive only scattered illumination from the tissue itself. This has the advantage of providing greater detail and allowing deeper veins to be better revealed. This technique is not the same as a structured light approach that is sometimes used to reveal depth information about objects hidden behind scattered light. This approach provides an increase in contrast. More complex pattern movement can be achieved by using more than two sources.

Note that this same technique can be used with the illumination design shown in FIG. 2. Rows, blocks, or individual sources can be turned on or off synchronously with the camera frame to optimize contrast. In particular, this can be done dynamically—when a potential vein is recognized, the LEDs over that vein can be turned off so the vein is illuminated by scattered light alone.

A second approach involves moving the optical pattern through mechanical means. This is not the preferred approach due to the design issues and additional complexity.

What is claimed is:

1. A non-contact illumination apparatus designed to be used in conjunction with a camera, display, and computing element(s) to reveal features such as veins based upon differential wavelength absorption beneath a biological surface being imaged whose underlying substrate, tissue, is a light scattering media, said illumination system comprising:
common optical focusing means, the same size as or larger than the surface to be illuminated;
an extended source means with its own optics such that the radiation from the source means fills or overfills the common optical focusing element;
where the source means is placed approximately at the optical focus of the common optical focusing means wherein the light exiting the common optical focusing means from any one point of the extended source is essentially parallel;
where the beam exiting the common optical focusing means is roughly perpendicular to the surface being illuminated;
where the plurality of all of the extended source means points exiting from the common optical focusing means form a common beam with controlled angular dispersion at the desired distance from the common optical focusing means wherein the beam meets the dual objectives of minimizing the scattering within the tissue by being roughly perpendicular to tissue layers and minimizing specular reflection from the surface of the tissue-structure by hitting the surface at multiple angles.

2. An apparatus of claim 1 wherein the main common optical means is a near spherical, asphere, or a spherical reflecting surface that collimates near infrared or visible light source means into a beam that covers the surface being imaged and strikes the surface at or near 90 degrees.

3. An apparatus of claim 2 where the light path is folded using one or more secondary mirrors to make a more compact package and/or remove the light sources away from the surface being irradiated;

4. An apparatus of claim 2 whose reflecting element is a dichroic reflective coating or other coating that transmits visible light and reflects infrared light onto the surface being imaged at or near 90 degrees wherein the surface is visible or partially visible through the reflector to the operator of the device;

5. An apparatus of claim 1 wherein the main common optical means is a lens or lens array that collimates near infrared or visible light source means into a beam that covers the surface being imaged and strikes the surface at or near 90 degrees.

6. An apparatus of claim 5 where the light path is folded using one or more secondary mirrors to make a more compact package and/or remove the light sources away from the surface being irradiated;

7. An apparatus of claim 6 where one or more of the secondary mirrors is composed of a series of parallel reflecting prisms to reflect the light in the desired direction wherein the volume required by such a reflector is much smaller that the volume required by a flat surface mirror.

8. The extended source of claim 1, where the extended source means is composed of multiple source elements, such source elements being LEDs, OLEDs, semiconductor lasers or the like assembled on to a surface in a pattern wherein that pattern being distributed away from the focal point of the common optical focusing means causes the light to hit the imaged surface at multiple angles around 90 degrees at any given point.

9. The extended source of claim 8 where individual sources mean can be controlled separately to vary the radiation output intensity wherein such control enables the radiation angle hitting the surface to be dynamically changed and enables the apparent position of the radiation source to be dynamically changed.

10. A method for changing the position and angular distribution of light by moving or selecting the light source synchronously with the frame rate of the camera and prior to the beginning of a new frame capture.

11. A method of claim 10 where the light sources are turned on and off in a position asymmetric way for removing imperfections in the optical system by using a computing element to find and replace scene elements that synchronously move with the light source change wherein such objects are defects in the optical system or structures above the surface such as hair that detract from the desired constant subsurface image.

12. A method of claim 10 to dynamically change the angular distribution of light impinging on the tissue surface to remove an angle that is causing a specular reflection.

13. A method of claim 10 to dynamically increase or decrease the angular distribution of light impinging on the tissue surface whereby the depth of tissue penetration is increased or decreased or whereby the reflection pattern of subcutaneous fat is changed to improve the contrast of a vein underneath such fat.

14. An apparatus of claim 1 which includes a light patterning means either as part of the existing optical element(s) or freestanding wherein such light patterning means illuminates patches or lines on the surface being imaged so that discrete areas of the surface are illuminated mainly by scattered light from the tissue of illuminated areas.

15. A light patterning means of claim 14 which is form through absorbing part of the light beam.

16. A light patterning means of claim 14 which is formed through reflecting part of the light beam into an area this is desired to be illuminated.

17. A light patterning means of claim 14 which is formed by concentrating areas of the beam through the use of lenses.

18. A method of moving the patterned light so that the illumination can be shifted such that some or all of the areas that were previously not illuminated are now illuminated and some or all of the areas that were previously illuminated now have lowered levels of illumination.

19. A method of claim 18 where the light sources are turned on and off in a position asymmetric way by moving or selecting the light source synchronously with the frame rate of the camera and prior to the beginning of a new frame capture wherein a frame can have a different light pattern than its predecessor.

20. A method of claim 14 which includes a computing means, such computing means removes the light patterning extracting the contrast information from the areas illuminated by scattered light.
